(19) United States
(12) Patent Application Publication
(10) Pub. No.: US 2024/0130869 A1
(43) Pub. Date: Apr. 25, 2024

(54) GRAFT DEPLOYMENT SYSTEMS AND METHODS

(71) Applicant: ACTIVAS Orthopedics, Inc., Coronado, CA (US)

(72) Inventors: Colin Murphy, Carlsbad, CA (US); Benjamin Arnold, San Diego, CA (US); Brian Bowman, Carlsbad, CA (US)

(21) Appl. No.: 18/382,899

(22) Filed: Oct. 22, 2023

Related U.S. Application Data
(60) Provisional application No. 63/418,870, filed on Oct. 24, 2022.

(51) Int. Cl. A61F 2/46; A61F 2/40

(52) U.S. Cl. CPC A61F 2/4612; A61F 2/40; A61F 2002/0072

(57) ABSTRACT

Systems, devices, and methods for deploying a graft to a treatment site of a patient. Exemplary systems include a device body and an insertion mechanism. The device body can have a handle, a trigger, and a controller. The insertion mechanism can be in operative association with the device body, and can have an outer sheath, a support shaft disposed at least partially within the outer sheath, an expansion mechanism coupled with the support shaft, and a graft in contact with the expansion mechanism.

### CROSS-REFERENCES TO RELATED APPLICATIONS

This application claims the benefit of U.S. Provisional Patent Application No. 63/418,870 filed Oct. 24, 2022, the disclosure of which is incorporated herein by reference.

### BACKGROUND

Embodiments of the present invention relate generally to graft deployment systems and methods, and in particular instances, to graft deployment systems and methods for rotator cuff repair and reconstruction.

Current graft deployment modalities enable surgeons to provide beneficial treatments to patients in need thereof. Yet still further improvements in graft deployment technology is desired. Embodiments of the present invention provide solutions to at least some of these outstanding needs.

### BRIEF SUMMARY

Exemplary graft deployment systems as disclosed herein can be provided as a single use device for the deployment of graft for rotator cuff reconstruction and rotator cuff repair. The device can include a handle, a trigger, and an insertion mechanism. The insertion mechanism consists of an outer sheath, a central shaft, and an expansion mechanism. The insertion mechanism can come preloaded with graft. To deploy the graft the trigger can be used to draw back the sheath and then expand the expansion mechanism, which presses the graft into position. The expansion mechanism can then be collapsed, and the insertion mechanism can be removed, leaving the graft in position for fixation.

Advantageously, embodiments of the present invention enable the performance of simplified surgical procedures by packaging full functionality into a single-use instrument, reducing cost associated with reprocessing. In some cases, preloading of the graft with the deployment tool can remove the need for manual preparation by the surgeon. The expansion feature can maintain rigidity after expansion, allowing for compression of the graft against target tissues, which is difficult with currently known solutions. Exemplary device embodiments simply procedures compared to some known solutions by minimizing the need for suturing.

Moreover, graft deployment systems disclosed herein do not require that a graft be prepared separately from the deployment system and then loaded by the physician into the deployment system. For example, the system can be provided for use by the physician or surgeon (or other individual) in a pre-loaded configuration, where the graft and the system are provided as a combination. Further, embodiments disclosed herein provide solutions where the system makes substantial contact with the graft during deployment, going beyond contact which is limited to edge contact or point contact. Further, embodiments disclosed herein provide solutions that do not require a frame around the edge of a graft for deployment, or that do not require the use of expanding point contacts, and hence can provide enhanced control of the center of a graft during deployment.

Still further, embodiments disclosed herein can be configured with an expansion mechanism which is semi-rigid, thus enabling the surgeon to have the ability to apply selective pressure to the graft during the fixation process, and also giving flexibility to the surgeon in terms of how they place fixation features. What is more, with pre-loaded graft system embodiments, the surgeon or user is not required to load the graft to the system during the operating procedure, thus enabling the surgeon to perform a more efficient procedure. Relatedly, with pre-loaded graft system embodiments, the surgeon or user does not need to perform the step of sizing the graft while performing the procedure. Relatedly, the graft sizing can be handled in the pre-operative period. This advantage can enable the surgeon to perform the surgery in a shorter amount of time, because they are not required to prepare the graft during the procedure. In another advantage, embodiments of the present invention encompass the use of an insertion mechanism which allows for the positioning of the graft with visualization of its depth and location prior to full deployment. In some cases, such visualization can be arthroscopic visualization. In some cases, such visualization can be ultrasound visualization. What is more, embodiments of the present invention employ the use of expansion mechanisms having a curved shape or profile.

In one aspect, embodiments of the present invention encompass graft deployment systems and methods for delivering a graft to a treatment site of a patient. Exemplary graft deployment systems can include a device body and an insertion mechanism. A device body can include a handle, a trigger, and a controller. An insertion mechanism can be in operative association with the device body, and can include an outer sheath, a support shaft disposed at least partially within the outer sheath, an expansion mechanism coupled with the support shaft, and a graft in contact with the expansion mechanism. In some cases, the expansion mechanism is a balloon expansion mechanism. In some cases, the expansion mechanism is a shape memory expansion mechanism. In some cases, the shape memory expansion mechanism is a nitinol expansion mechanism. The expansion mechanism can have one or more cutouts. In some cases, the trigger operates to retract the outer sheath in a proximal direction. In some cases, the trigger operates to expand the expansion mechanism. The controller can include a lever, a button, or the like. In some cases, the controller operates to contract the expansion mechanism. In some cases, a system may further include a fluid source that provides a fluid to the expansion mechanism.

Exemplary methods for delivering a graft to a treatment site of a patient can include positioning a distal portion of a graft deployment system at the treatment site of the patient, actuating a trigger of the graft deployment system, and delivering the graft to the treatment site of the patient. In some cases, the graft deployment system includes an outer sheath, the graft, and an expansion mechanism supporting the graft, and the outer sheath operates to maintain the expansion mechanism in a constrained configuration. In some cases, actuation of the trigger operates to retract the outer sheath of the graft deployment system and thereby unconstrain the expansion mechanism. In some case, the step of delivering the graft to the treatment site of the patient can occur after the expansion mechanism has transitioned to an unconstrained configuration. In some cases, the expansion mechanism is a balloon expansion mechanism. In some cases, the expansion mechanism is a shape memory expansion mechanism. In some cases, the shape memory expansion mechanism is a nitinol expansion mechanism. The expansion mechanism can have one or more cutouts. In some cases, actuation of the trigger operates to retract the outer sheath in a proximal direction. In some cases, actuation of the trigger operates to expand the expansion mechanism. In some cases, methods can include actuating a controller of the graft deployment system to contract the expansion mechanism. In some cases, methods can include providing a fluid to the expansion mechanism. In some cases, the treatment site of the patient is a rotator cuff of the patient.

### BRIEF DESCRIPTION OF THE DRAWINGS

Inventive features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates aspects of a graft deployment system, according to embodiments of the present invention;

FIG. 2 illustrates aspects of a graft deployment system, according to embodiments of the present invention;

FIG. 3 illustrates aspects of a graft deployment system, according to embodiments of the present invention;

FIGS. 4A and 4B illustrate aspects of a graft deployment system, according to embodiments of the present invention;

FIG. 5 illustrates aspects of a graft deployment system, according to embodiments of the present invention;

FIG. 6 illustrates aspects of a graft deployment system, according to embodiments of the present invention;

FIGS. 7A, 7B, and 7C illustrate aspects of a graft deployment system, according to embodiments of the present invention;

FIG. 8 illustrates aspects of a graft deployment system, according to embodiments of the present invention;

FIGS. 9A and 9B illustrate aspects of a graft deployment system, according to embodiments of the present invention;

FIGS. 10A, 10B, and 10C illustrate aspects of a graft deployment system, according to embodiments of the present invention;

FIG. 11 illustrates aspects of a graft deployment system, according to embodiments of the present invention;

FIG. 12 illustrates aspects of a graft deployment system, according to embodiments of the present invention;

FIGS. 13A and 13B illustrate aspects of a graft deployment system, according to embodiments of the present invention;

FIG. 14 illustrates aspects of a graft deployment system, according to embodiments of the present invention;

FIGS. 15A, 15B, and 15C illustrate aspects of a graft deployment system, according to embodiments of the present invention;

FIG. 16 illustrates aspects of a graft deployment system, according to embodiments of the present invention;

FIGS. 17A and 17B illustrate aspects of a graft deployment system, according to embodiments of the present invention;

FIGS. 18A, 18B, and 18C illustrate aspects of a graft deployment system, according to embodiments of the present invention;

FIGS. 19A and 19B illustrate aspects of a graft deployment system, according to embodiments of the present invention;

FIGS. 20A and 20B illustrate aspects of a graft deployment system, according to embodiments of the present invention;

FIG. 21 illustrates aspects of a graft deployment system, according to embodiments of the present invention;

FIG. 22 illustrates aspects of a graft deployment system, according to embodiments of the present invention;

FIGS. 23A and 23B illustrate aspects of a graft deployment system, according to embodiments of the present invention;

FIGS. 24A and 24B illustrate aspects of a graft deployment system, according to embodiments of the present invention;

FIG. 25 illustrates aspects of a graft deployment system, according to embodiments of the present invention; and

FIGS. 26A and 26B illustrate aspects of a graft deployment system, according to embodiments of the present invention.

### DETAILED DESCRIPTION

Specific embodiments of the disclosed device, system and method of use will now be described with reference to the drawings. Nothing in this detailed description is intended to imply that any particular component, feature, or step is essential to embodiments of the invention.

Graft deployment systems disclosed herein can be used to deploy grafts to any of a variety of patient treatment sites. In some cases, a graft deployment system can be used to deploy a graft to a rotator cuff of a patient. In some cases, a graft deployment system can be used to deploy a graft for an Achilles tendon repair, a patella tendon repair, a triceps repair, a quadriceps repair, or the like.

Graft deployment systems disclosed herein can be provided with a variety of different expansion mechanisms, including balloon expansion mechanisms, shape memory expansion mechanisms, and the like. For example, a graft deployment system can employ the use of balloon during placement or deployment of a graft. In some cases, a shape memory expansion mechanism can include a nitinol wire that is coiled in a sheet and curled. In some embodiments, an expansion mechanism can be configured with a shape that conforms with a particular patient tissue or anatomical feature.

Balloon Expansion Mechanism

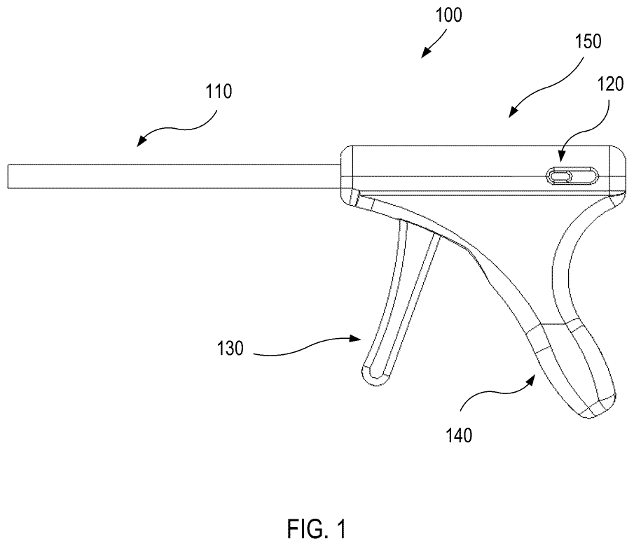

Turning now to the drawings, FIG. 1 depicts aspects of a graft deployment system 100, according to embodiments of the present invention. As shown here, system 100 is illustrated in an initial state, and includes an insertion mechanism 110, a lever 120, a trigger 130, and a handle 140. The handle is held such that the trigger can be pulled comfortably. In some cases, the handle 140, trigger 130, and lever 120 can be parts of a device body 150. In some cases, the insertion mechanism 110 can include an outer sheath, a central shaft, and an expansion mechanism. In some cases, the central shaft of the insertion mechanism 110 can be coupled with or affixed to the device body 150. In some cases, the central shaft of the expansion mechanism can be affixed to the device body. In some cases, a system or device body can include a button instead of a lever. In some cases, a system or device body can include some other type of controller instead of a lever.

In some cases, the lever 120 can be used for retracting the expansion mechanism. In some cases, a system can include a button or other control mechanism, instead of a lever. In some cases, a handle 140 can be provided in a pistol grip configuration, as shown here. In some cases, a handle can be provided in a linear orientation configuration. In some cases, the lever 120 can be integrated or built into the trigger 130.

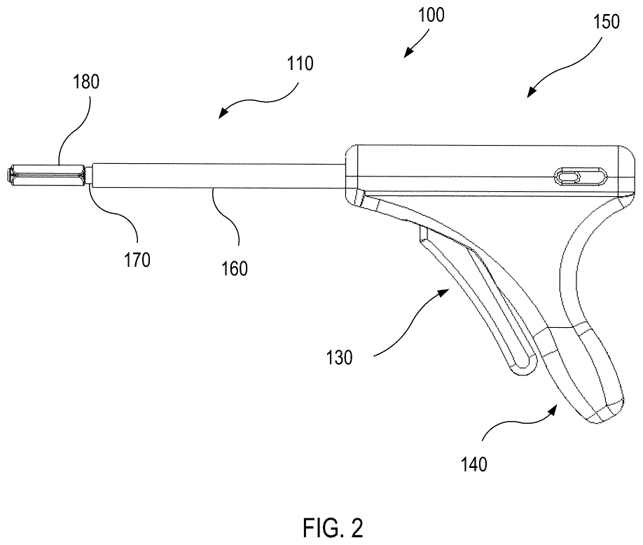

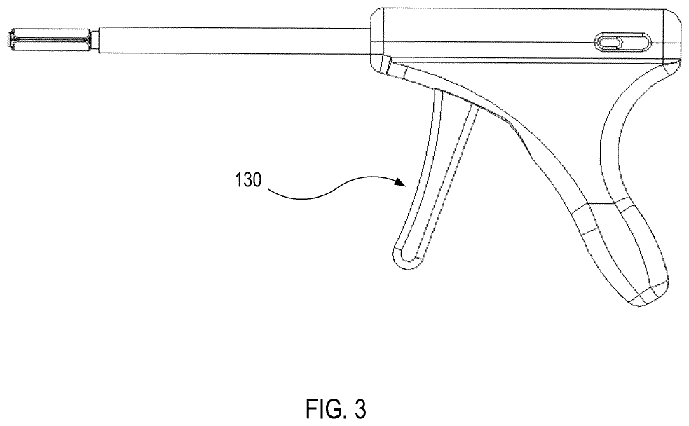

FIG. 2 depicts the system 100 in a subsequent state. As shown here, the trigger 130 can be pulled toward the handle 140, thus causing a sheath 160 of the insertion mechanism 110 to retract (e.g. toward the device body 150), thereby exposing the central or support shaft 170 and a graft 180. As shown in FIG. 3, the trigger 130 can be released.

In exemplary balloon expansion mechanism embodiments, the trigger is double action, with a mechanism interior to the handle which can permanently switch functions once the trigger is pulled. The first time the trigger is pulled the outer sheath of the insertion mechanism can be drawn back to reveal the graft. The second time the trigger is pulled it can actuate a piston inside of the handle, filling the balloon with saline. The lever on the handle can be used to draw back the piston, removing the saline and deflating the balloon for removal.

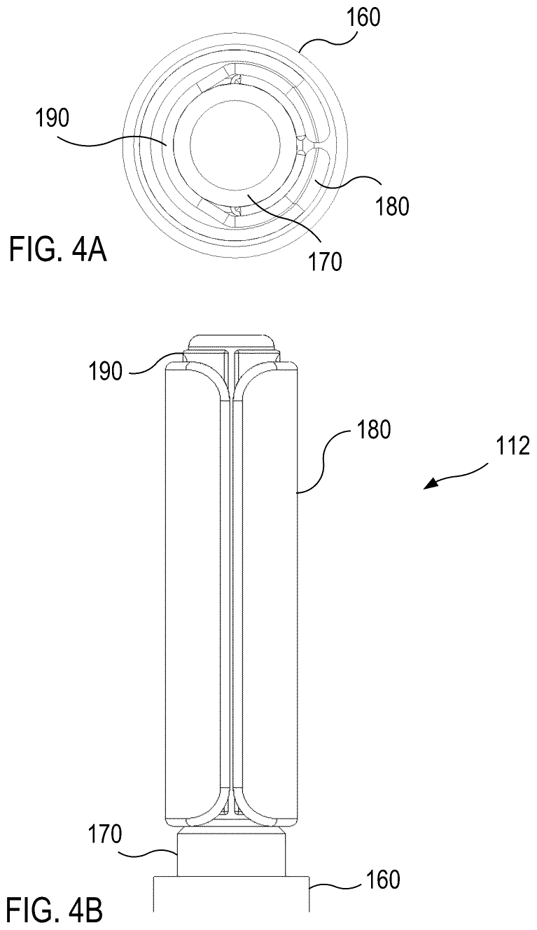

FIG. 4A shows an end view of a distal portion of the system, illustrating the outer sheath 160, the graft 180, the central shaft 170, and a balloon expansion mechanism 190. FIG. 4B shows a side view of the distal portion 112 of the insertion system, illustrating the outer sheath 160 (in a retracted position), the central shaft 170, the graft 180, and the balloon expansion mechanism 190.

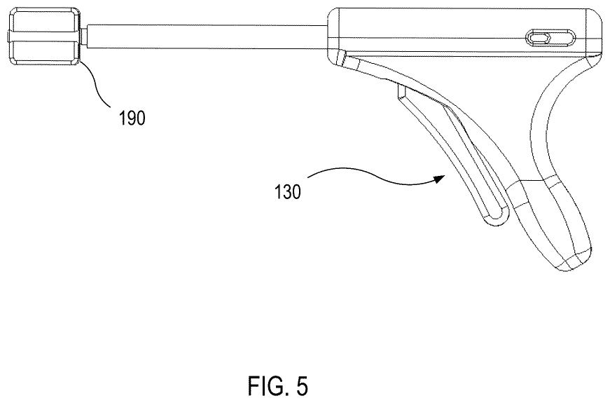

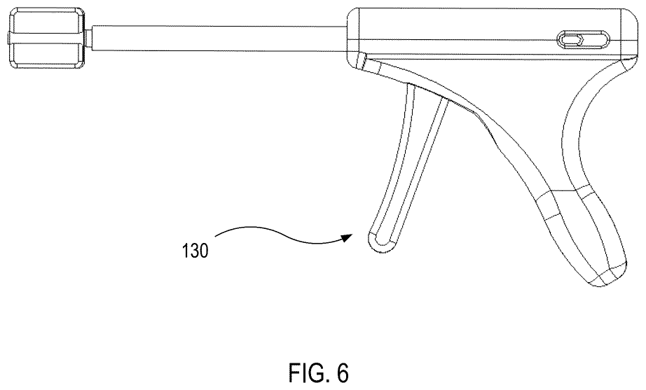

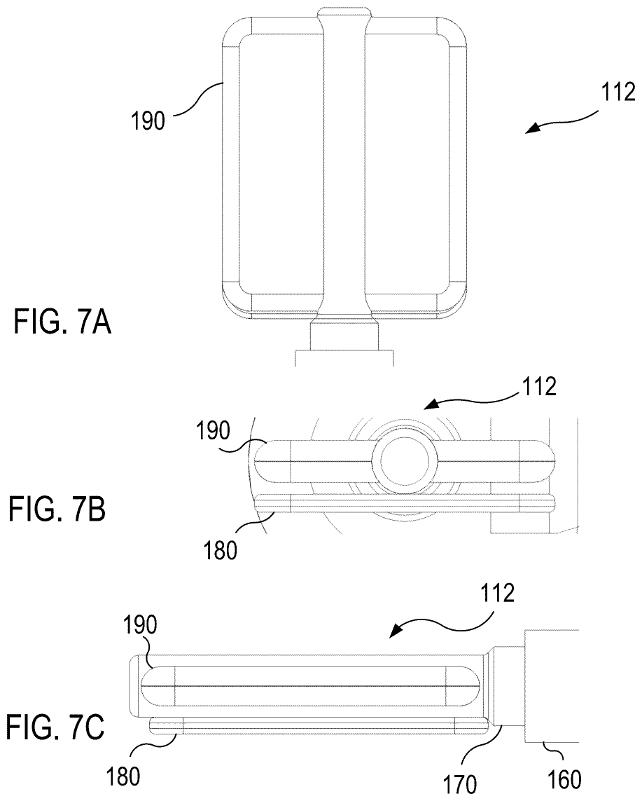

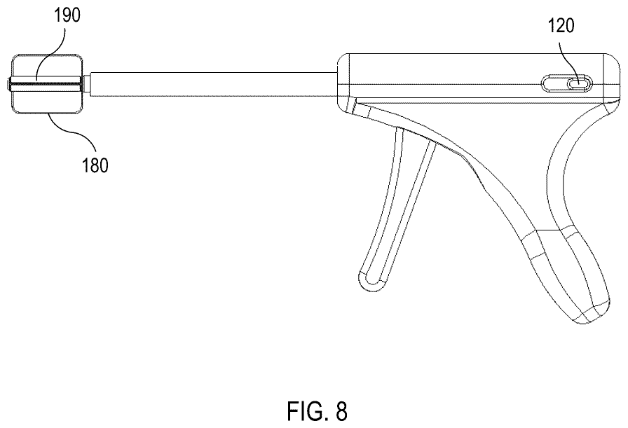

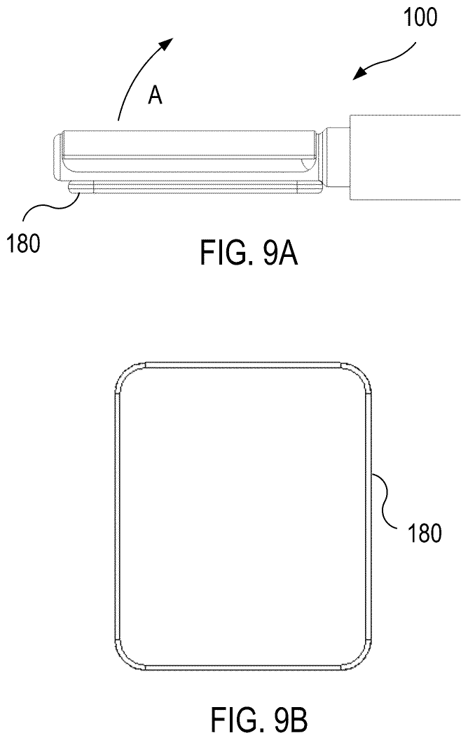

As shown in FIG. 5, the trigger 130 can then be pulled or activated, thus causing the balloon expansion mechanism 190 to expand. As shown in FIG. 6, the trigger 130 can then be released. FIG. 7A provides a top view of a distal portion 112 of the insertion system, FIG. 7B provides an end view of distal portion 112, and FIG. 7C provides a side view of distal portion. As shown here, distal portion 112 includes a balloon expansion mechanism 190 and a graft 180. It is then possible to pull or activate the lever 120, thus causing the balloon expansion mechanism 190 to deflate, as shown in FIG. 8. The system 100 can then be removed or moved away from the patient treatment site, as indicated by arrow A in the side view of FIG. 9A, thus leaving the graft 180 in place at the treatment site or location, as shown in the top view of FIG. 9B.

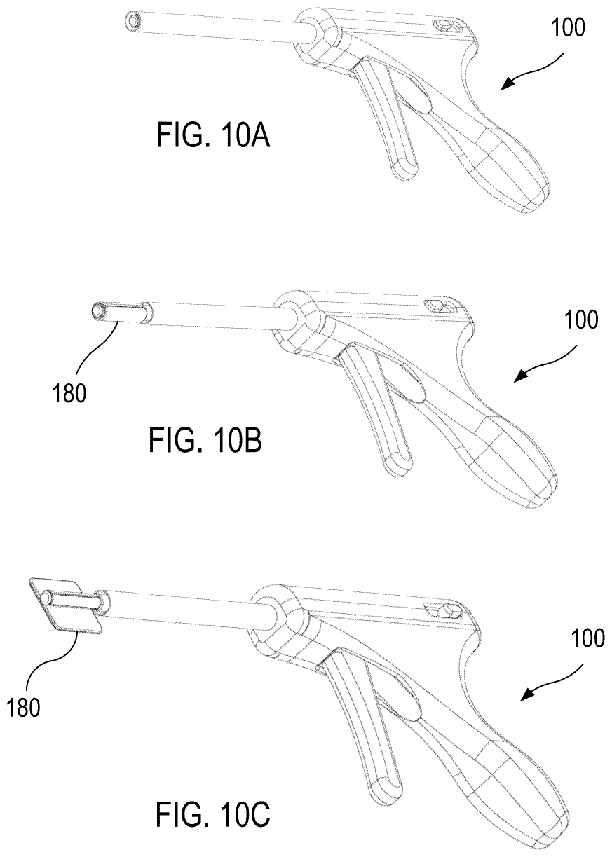

FIGS. 10A to 10C illustrate a sequence of steps that can be performed using the system 100 to deploy a graft 180.

In some embodiments, a balloon expansion mechanism can include a balloon that is inflated with air. In some embodiments, a balloon expansion mechanism can include a balloon that is inflated with saline. Balloon expansion mechanisms can be configured to be inflated with any desired fluid, and embodiments of the present invention encompass the use of any desired fluid source. In some cases, a balloon expansion mechanism may be constrained only from its proximal end. In some embodiments, a balloon expansion mechanism can have a curved shape. In some cases, a graft deployment system with a balloon expansion mechanism can include a single trigger, or in some cases, multiple triggers.

According to some embodiments, at the distal end of the insertion mechanism the central shaft supports the expansion mechanism, which wraps around and rests in the central shaft when not in an expanded state. In balloon expansion mechanism embodiments, the central shaft can be canulated. The graft can be preloaded wrapped around the central shaft and the expansion mechanism, with the outer sheath covering the graft and other components radially.

Shape Memory Expansion Mechanism

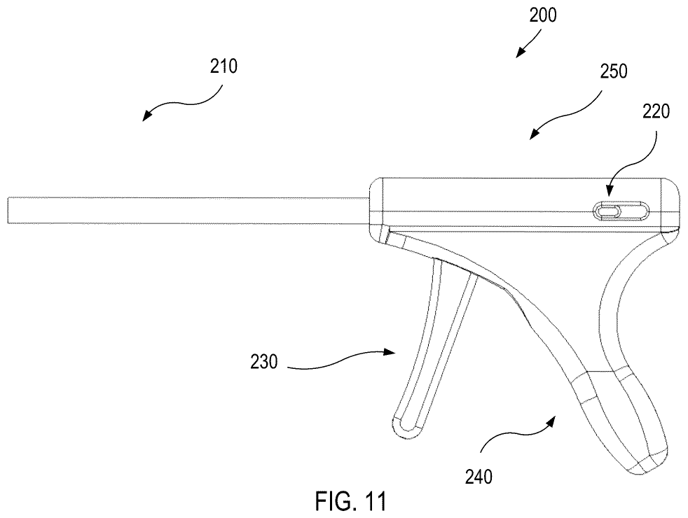

FIG. 11 depicts aspects of a graft deployment system 200, according to embodiments of the present invention. As shown here, system 200 is illustrated in an initial state, and includes an insertion mechanism 210, a lever 220, a trigger 230, and a handle 240. The handle is held such that the trigger can be pulled comfortably. In some cases, the handle 240, trigger 230, and lever 220 can be parts of a device body 250. In some cases, the insertion mechanism 210 can include an outer sheath, a central shaft, and an expansion mechanism. In some cases, the central shaft of the insertion mechanism 210 can be coupled with or affixed to the device body 250. In some cases, the central shaft of the expansion mechanism can be affixed to the device body. In exemplary shape-memory expansion mechanism embodiments, the trigger is single action. Pulling the trigger once will draw back the outer sheath of the insertion mechanism, revealing the graft and allowing the nitinol expansion feature to expand. The lever on the handle can be used to reextend the outer sheath, compressing the shape memory expansion mechanism.

In some cases, the lever 220 can be used for retracting the expansion mechanism. In some cases, a system can include a button or other control mechanism, instead of a lever. In some cases, a handle 240 can be provided in a pistol grip configuration, as shown here. In some cases, a handle can be provided in a linear orientation configuration. In some cases, the lever 220 can be integrated or built into the trigger 230.

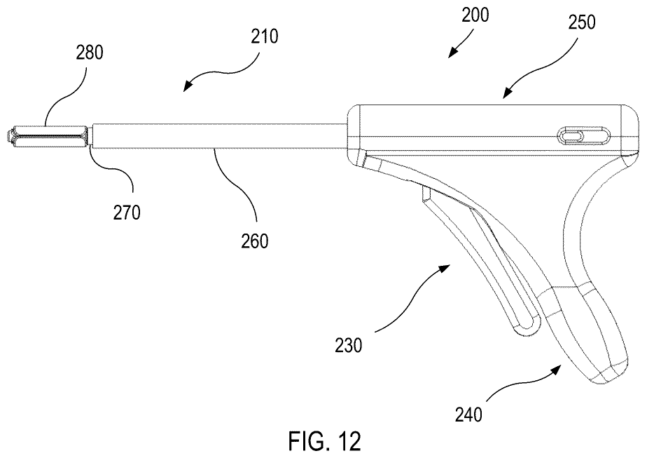

FIG. 12 depicts the system 200 in a subsequent state. As shown here, the trigger 230 can be pulled toward the handle 240, thus causing a sheath 260 of the insertion mechanism 210 to retract (e.g. toward the device body 250), thereby exposing the central shaft 270 and a graft 280.

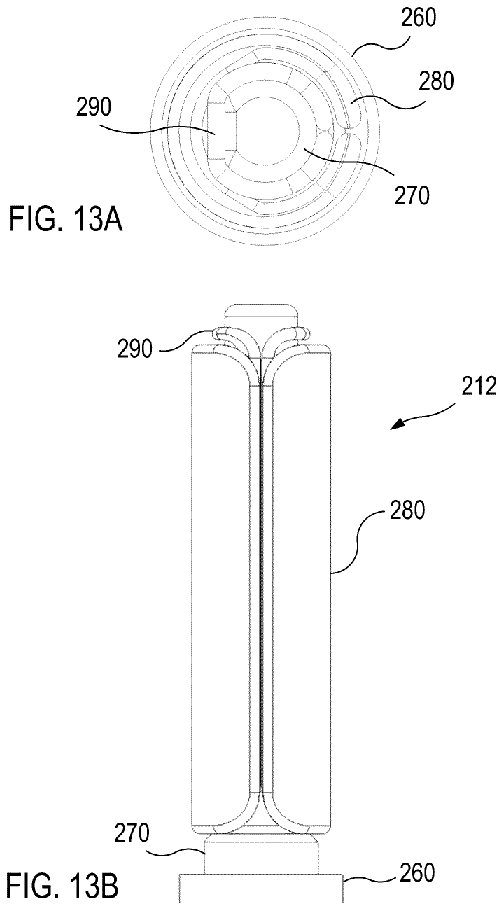

FIG. 13A shows an end view of a distal portion of the system, illustrating the outer sheath 260, the graft 280, the central shaft 270, and a shape memory expansion mechanism 290. FIG. 13B shows a side view of the distal portion 212 of the insertion system, illustrating the outer sheath 260 (in a retracted position), the central shaft 270, the graft 280, and the shape memory expansion mechanism 290. In exemplary embodiments, the shape memory expansion mechanism 290 includes a shape memory alloy, such as nickel-titanium (Nitinol) or copper-aluminum-nickel.

In some cases, an expansion mechanism can include a material that is capable of stretching significantly without permanent deformation, and that exhibits insignificant or no shape memory characteristics (e.g. changing shape upon application of heat) particularly at room temperature. For example, an expansion mechanism can include superelastic nitinol.

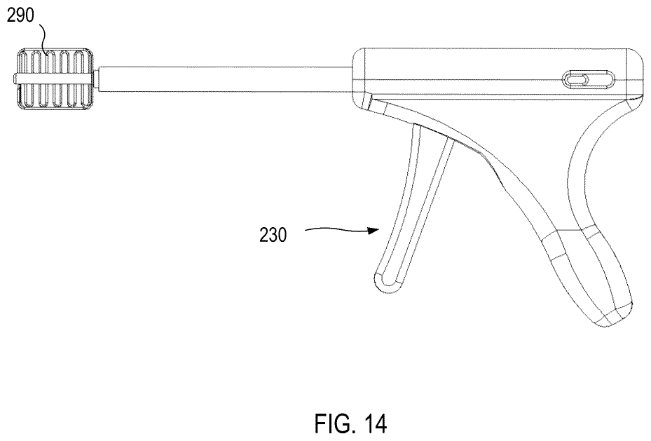

As shown in FIG. 14, the trigger 230 can then be released, thus causing the shape memory expansion mechanism 290 to expand (e.g. upon warming or heating).

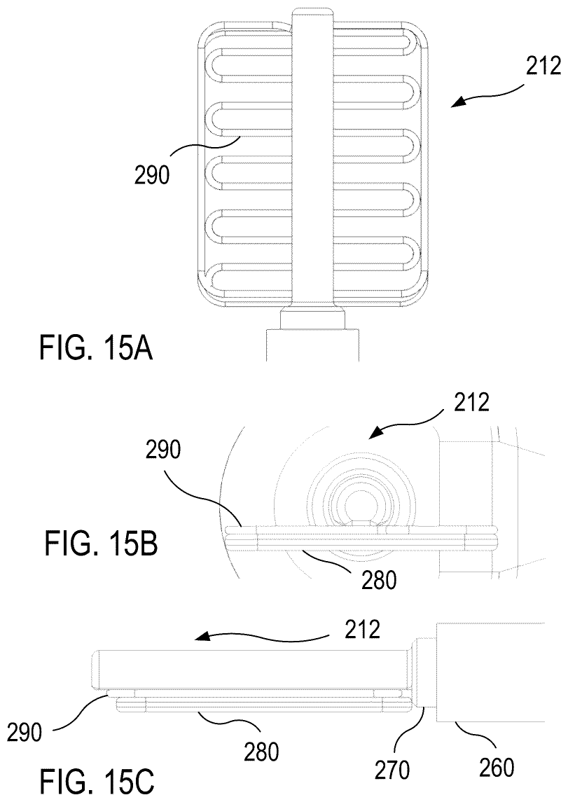

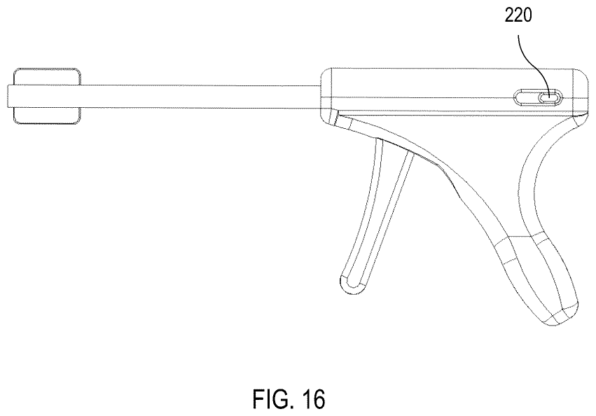

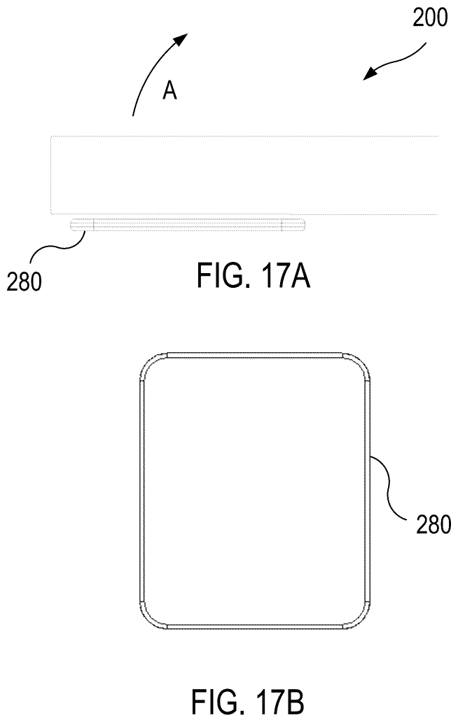

FIG. 15A provides a top view of a distal portion 212 of the insertion system, FIG. 15B provides an end view of distal portion 212, and FIG. 15C provides a side view of distal portion. As shown here, distal portion 212 includes a shape memory expansion mechanism 290 and a graft 280. It is then possible to pull or activate the lever 220, thus causing the sheath to extend distally, thereby causing the shape memory expansion mechanism 290 to compress, as shown in FIG. 16. The system 200 can then be removed or moved away from the patient treatment site, as indicated by arrow A in the side view of FIG. 17A, thus leaving the graft 280 in place at the treatment site or location, as shown in the top view of FIG. 17B.

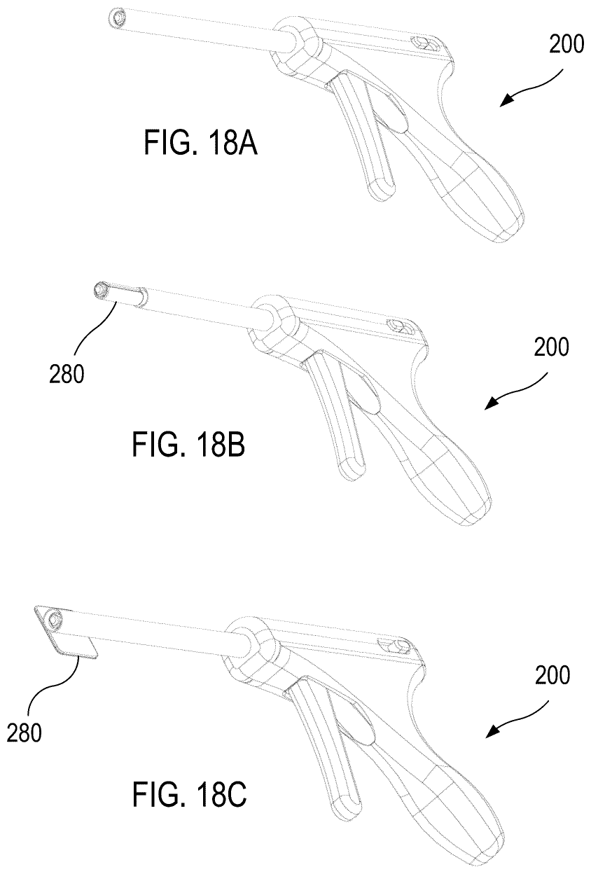

FIGS. 18A to 18C illustrate a sequence of steps that can be performed using the system 200 to deploy a graft 280.

According to some embodiments, at the distal end of the insertion mechanism the central shaft supports the expansion mechanism, which wraps around and rests in the central shaft when not in an expanded state. The graft can be preloaded wrapped around the central shaft and the expansion mechanism, with the outer sheath covering the graft and other components radially.

### Related Embodiments

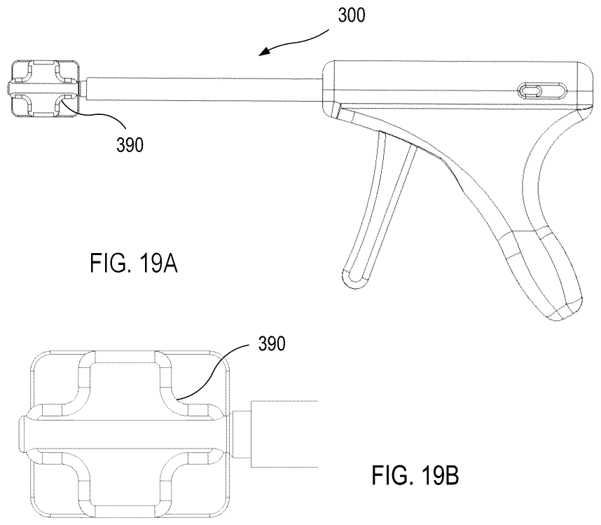

FIGS. 19A and 19B illustrate a graft deployment system 300 embodiment having an expansion mechanism 390 with cutouts. In some cases, an expansion mechanism can have windows cut to provide visualization of the graft or openings for the application of fixation.

According to some embodiments, a graft can be provided as a dermal graft, a collagen graft, a synthetic textile graft, or the like.

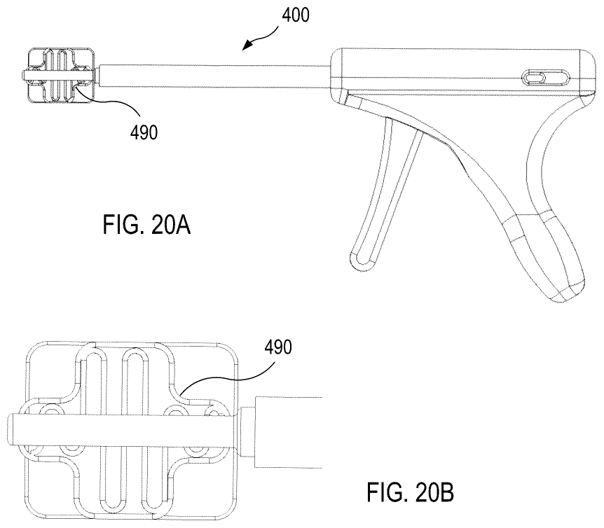

FIGS. 20A and 20B illustrate a graft deployment system 400 embodiment having an expansion mechanism 490 with cutouts. In some cases, an expansion mechanism can have windows cut to provide visualization of the graft or openings for the application of fixation.

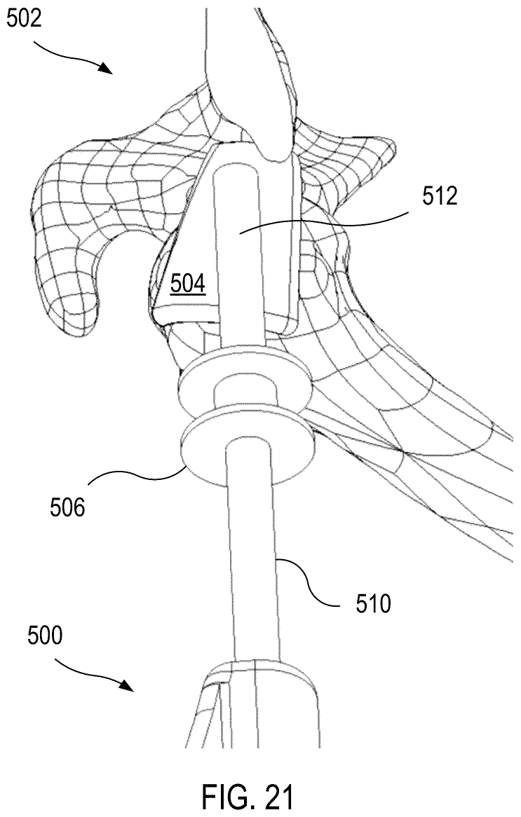

FIG. 21 depicts aspect of a procedure whereby a graft deployment system 500 can be used to deploy a graft at a treatment site of a patient. In this embodiment, the treatment site is a shoulder 502 of the patient, and the graft is deployed at a rotator cuff 504 of the shoulder. An inserter mechanism 510 of the system 500 can be inserted through a portal mechanism 506, so as to access a space within the patient's shoulder. As shown here, a distal portion 512 of the insertion mechanism 510 is positioned at the treatment site near the rotator cuff 504.

Hence, use of a graft deployment system can involve advancing an insertion mechanism through a portal, toward the desired or proper position. The insertion mechanism can be inserted to a desired depth of the graft and positioned so that the center of the graft is at the center of the desired graft location. Once the desired location is reached, the trigger can be pulled, retracting the outer sheath. For balloon expansion mechanism embodiments, the trigger can be pulled a second time to expand the balloon. The expansion mechanism, now expanded, can be used to tamp down the graft, and to hold the graft in place while fixation is applied to the graft (e.g. to a distal end of the graft). Once sufficient fixation is applied, the lever can be used to compress the expansion feature, and the graft deployment system can be removed from the patient.

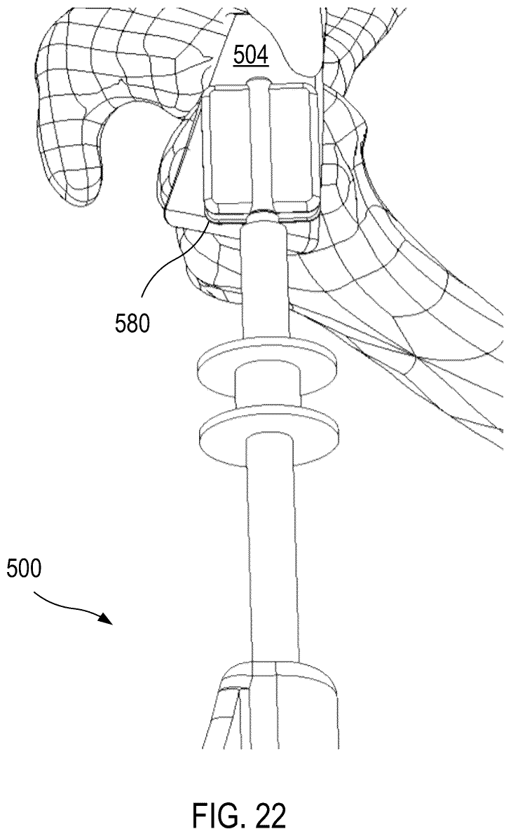

FIG. 22 illustrates expansion of the graft 580 as the graft is deployed by the system 500 at the treatment site (e.g. adjacent the rotator cuff 504).

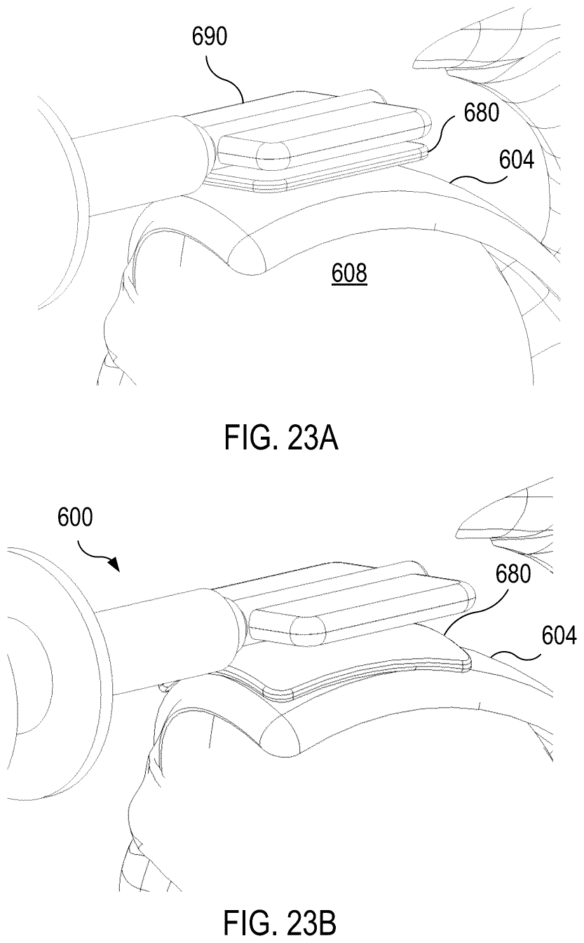

FIG. 23A depicts an initial placement of a graft 680 (e.g. at the rotator cuff 604) near the patient bone 608, as the expansion mechanism 690 is activated or expanded. Once the graft 680 is positioned in the initial location, the graft 680 can be pressed, moved, or otherwise manipulated (e.g. with system 600, or with a tool—not shown) so that the shape of the graft 680 conforms with the shape of the nearby tissue or anatomical structure (e.g. rotator cuff 604), as illustrated in FIG. 23B.

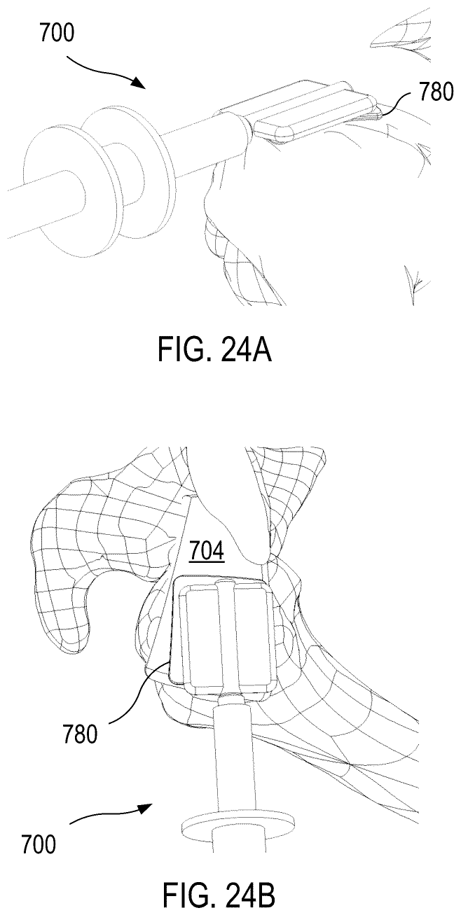

As shown in FIGS. 24A and 24B, a graft deployment system 700 can be rotated to tamp down a graft 780 (e.g. against a patient tissue or anatomical structure, such as a rotator cuff 704).

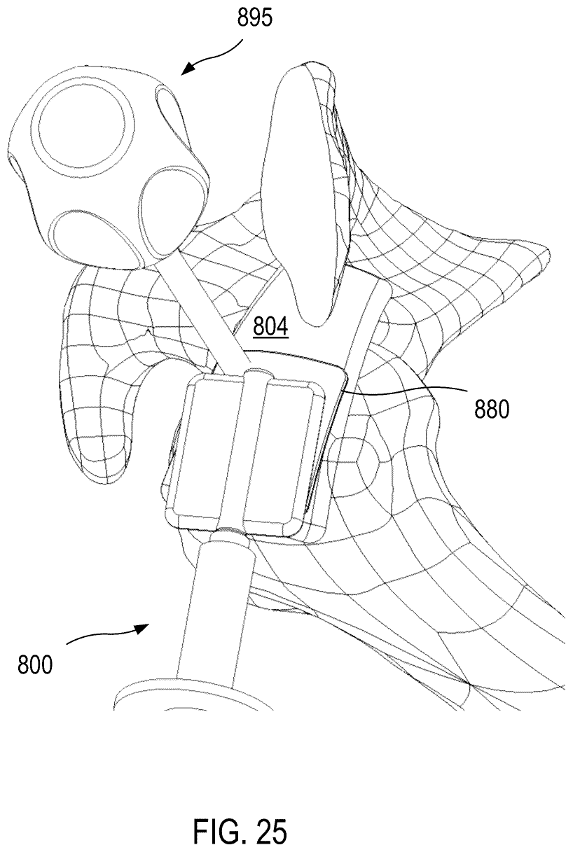

As shown in FIG. 25, a graft deployment system 800 can be used to hold a graft 880 in place while fixation is applied (e.g. by a fixation instrument 895), so as to fix the graft 880 with a patient tissue or anatomical structure (e.g. such as a rotator cuff 804).

A graft deployment system can be used to deploy grafts of any desired size. In some cases, systems can be provided in various sizes according to the required or desired sizes of the graft. In some cases, two size configurations may be provided, one size for repair procedures and another size for reconstruction procedures. In graft deployment system embodiments for repair procedures, a graft can have size dimensions of about 20-25 mm by 25-30 mm and can be about 1-3 mm thick, and the outer sheath diameter can be about 10 mm. In graft deployment system embodiments for reconstruction procedures, a graft can have size dimensions of about 40-75 mm by 40-75 mm and can be about 3-5 mm thick, and the outer sheath diameter can be about 12 mm or larger.

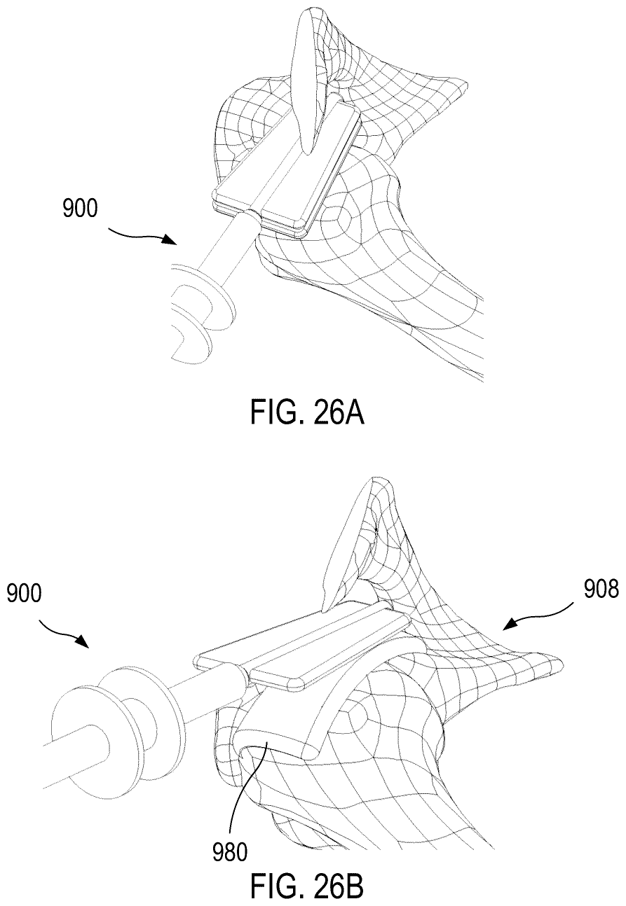

As shown in FIGS. 26A and 26B, a graft deployment system 900 can be used deploy a large graft 980 which is used for reconstruction. FIG. 26B depicts the graft 980 after it has been tamped down by the system 900 against the patient tissue or anatomical structure (e.g. bone 908).

In exemplary deployment system embodiments, one or more components of the system can be manufactured from or include a plastic material. In some cases, a pre-loaded graft and deployment system can be preloaded with suture through the graft.

Although the preceding description contains significant detail in relation to certain preferred embodiments, it should not be construed as limiting the scope of the invention but rather as providing illustrations of the preferred embodiments.

Embodiments of the present invention encompass kits having one or more components of a system as disclosed herein. In some embodiments, the kit includes one or more system components, along with instructions for using the component(s) for example according to any of the methods disclosed herein.

All features of the described systems and devices are applicable to the described methods mutatis mutandis, and vice versa.

In addition, each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present disclosure have been shown and described herein, it will be understood to those skilled in the art that such embodiments are provided by way of example only. Numerous variations, changes, and substitutions will now occur to those skilled in the art without departing from embodiments of the present invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A graft deployment system, comprising:
a device body having a handle, a trigger, and a controller; and
an insertion mechanism in operative association with the device body, the insertion mechanism having an outer sheath, a support shaft disposed at least partially within the outer sheath, an expansion mechanism coupled with the support shaft, and a graft in contact with the expansion mechanism.

2. The graft deployment system according to claim 1, wherein the expansion mechanism is a balloon expansion mechanism.

3. The graft deployment system according to claim 1, wherein the expansion mechanism is a shape memory expansion mechanism.

4. The graft deployment system according to claim 1, wherein the expansion mechanism is a nitinol expansion mechanism or a superelastic nitinol expansion mechanism.

5. The graft deployment system according to claim 1, wherein the expansion mechanism has one or more cutouts or a curved shape.

6. The graft deployment system according to claim 1, wherein the trigger operates to retract the outer sheath in a proximal direction.

7. The graft deployment system according to claim 1, wherein the trigger operates to expand the expansion mechanism.

8. The graft deployment system according to claim 1, wherein the controller comprises a member selected from the group consisting of a lever and a button.

9. The graft deployment system according to claim 1, wherein the controller operates to contract the expansion mechanism.

10. The graft deployment system according to claim 1, further comprising a fluid source that provides a fluid to the expansion mechanism.

11. The method of delivering a graft to a treatment site of a patient, the method comprising:
positioning a distal portion of a graft deployment system at the treatment site of the patient, the graft deployment system comprising an outer sheath, the graft, and an expansion mechanism supporting the graft, wherein the outer sheath operates to maintain the expansion mechanism in a constrained configuration;
actuating a trigger of the graft deployment system to retract the outer sheath of the graft deployment system and thereby unconstrain the expansion mechanism;
delivering the graft to the treatment site of the patient after the expansion mechanism has transitioned to an unconstrained configuration.

12. The method according to claim 11, wherein the expansion mechanism is a balloon expansion mechanism.

13. The method according to claim 11, wherein the expansion mechanism is a shape memory expansion mechanism.

14. The method according to claim 11, wherein the shape memory expansion mechanism is a nitinol expansion mechanism or a superelastic nitinol expansion mechanism.

15. The method according to claim 11, wherein the expansion mechanism has one or more cutouts or a curved shape.

16. The method according to claim 11, wherein the trigger operates to retract the outer sheath in a proximal direction.

17. The method according to claim 11, wherein the trigger operates to expand the expansion mechanism.

18. The method according to claim 11, comprising actuating a controller of the graft deployment system to contract the expansion mechanism.

19. The method according to claim 11, further comprising providing a fluid to the expansion mechanism.

20. The method according to claim 11, wherein the treatment site of the patient is a rotator cuff of the patient.
